(19) United States
(12) Patent Application Publication
(10) Pub. No.: US 2013/0018339 A1
(43) Pub. Date: Jan. 17, 2013

(54) Disposable Absorbent Article Design Utilizing Unbleached Pulp Fibers for the Absorbent Core

(71) Applicants: Tom Kaiser, Marion, OH (US); Steven D. Linton, Snellville, GA (US)

(72) Inventors: Tom Kaiser, Marion, OH (US); Steven D. Linton, Snellville, GA (US)

(73) Assignee: Associated Hygienic Products LLC, Duluth, GA (US)

(21) Appl. No.: 13/181,339

(22) Filed: Jul. 12, 2011

(51) Int. Cl. A61L 15/56; B23P 17/00; A61L 15/22

(52) U.S. Cl. 604/361; 604/367; 604/374; 29/419.1

(57) ABSTRACT

Absorbent articles and methods for making absorbent articles incorporate unbleached fibers in an absorbing core. Example embodiments provide unbleached fibers which are totally chlorine free. Such unbleached fibers provide features and advantages not present or recognized over prior articles. According to one embodiment, a disposable absorbent article having a front waist region, a back waist region, and a crotch region located between the front and back waist regions, is provided. The disposable absorbent article includes a liquid pervious topsheet and a liquid impervious backsheet. Additionally, the disposable absorbent article includes an absorbent core disposed between at least a portion of the topsheet and bottom sheet. This absorbent core comprises a superabsorbent material and pulp material where at least a portion of the pulp material includes unbleached fibers having constituents which alter the performance properties of the absorbent core.

### TECHNICAL FIELD

The present application relates generally to disposable absorbent garments and, more particularly, to a disposable absorbent garment having an absorbent core comprising unbleached pulp and a method of making the same.

### BACKGROUND

Disposable absorbent garments contemplated by the present application include disposable diapers, disposable pull-on garments, adult incontinent articles, sanitary napkins and the like. These garments are worn about the lower torso or waist of the user so as to receive and contain urine and other bodily wastes. The principal elements of a typical disposable absorbent garment include a liquid permeable inner layer that is closest to the wearer (or topsheet), a liquid impermeable outer layer (or backsheet) and an absorbent core, sandwiched between the inner and outer layers. The topsheet functions to wick moisture away from the wearer. The absorbent core absorbs and stores the urine and other waste. The backsheet functions to protect the exterior environment from urine and waste. These layers may be combined with other materials, layers, or features to specifically adapt the disposable absorbent garment to a particular purpose or to enhance the activity of the disposable absorbent garment. Elastic members may be incorporated into a part or area of the garment to provide correct fitting or sealing of the garment to the user, for example.

As the usage of disposable absorbent garments has expanded, their complexity has increased with the incorporation of additional features serving to enhance their performance and appearance. As a result, the costs of materials and the complexity of manufacturing processes have increased in conjunction with this increase in complexity.

For example, currently when manufacturing an absorbent core for a baby diaper, designs incorporate a bleached or white fiber pulp. Many times this bleached fiber is an elemental chlorine-free (ECF) material or a totally chlorine-free (TCF) material which does not utilize chlorine in the whitening process. In the current state of the art, it is believed that such a bleached fiber material is advantageous both from a performance and from a cosmetic standpoint. Specifically, it is believed that long and coarse bleached fiber produced from southern softwoods provides better fluid distribution as opposed to an unbleached fiber which generally also includes undesirable constituents (e.g., lignin, hemicelluloses, sugars, hardwood fibers, softwood fibers, other organics and the like). Further, bleached or whitened fiber materials are preferred from a marketability standpoint because the whiteness of a baby diaper gives a perception of hygienic cleanliness.

### SUMMARY

The present application provides for absorbent articles and methods for making absorbent articles which incorporate unbleached fibers in an absorbing core. Example embodiments provide unbleached fibers which are totally chlorine free. The construction and use of such unbleached fibers provide features and advantages not present or recognized over prior unbleached fibers.

According to one embodiment, a disposable absorbent article having a front waist region, a back waist region, and a crotch region located between the front and back waist regions is provided. The disposable absorbent article includes a liquid pervious topsheet and a liquid impervious backsheet. Additionally, the disposable absorbent article includes an absorbent core disposed between at least a portion of the topsheet and bottom sheet. This absorbent core comprises a superabsorbent material and pulp material where at least a portion of the pulp material includes unbleached fibers. In some embodiments, the unbleached fiber includes excess constituents with respect to normal bleached fiber which alter the absorbent properties of the absorbent core.

According to another embodiment, a method of making a disposable absorbent article having a front waist region, a back waist region, and a crotch region located between the front and back waist regions is provided. The method includes forming an absorbent core, where the absorbent core comprises a majority part superabsorbent material and a minority part pulp material which includes unbleached fibers. The absorbent core is then placed between a liquid pervious topsheet and a liquid impervious backsheet. Additionally, the topsheet and backsheet are coupled along the front waist region, back waist region and crotch region.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 illustrates a disposable absorbent garment in accordance with an embodiment of the present application; and

FIG. 2 illustrates a cross-section of one embodiment of an absorbent diaper in accordance with an embodiment of the present application.

### DETAILED DESCRIPTION

As described previously, various aspects of the present invention are particularly suited to or for a disposable absorbent garment, such as baby diapers or training pants, for example. To illustrate the invention and preferred embodiments of the invention, much of the following Detailed Description will be provided in the context of such disposable absorbent garments. It is contemplated that various aspects of the inventive composite, garment, system and process may be applicable to other material structures and processes. This Detailed Description and exemplary embodiment should not, therefore, be construed as limiting the invention to the structures, configurations, methods, and processes described herein.

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or article described herein can be implemented with respect to any other method or article described herein.

As used herein, “disposable absorbent garment” or “disposable absorbent article” refers to a disposable garment which absorbs and contains body exudates. Typically, the garments are intended to be discarded after a limited period of use. Exemplary embodiments of disposable absorbent garments are diapers and training pants, adult incontinent products and sanitary napkins.

As used herein, “absorbent core” refers to the materials of the absorbent garment that are intended to provide the primary absorbency capabilities of the article.

As used herein, the term “inner” refers to a surface that faces the wearer when in use.

As used herein, the term “outer” refers to a surface opposite that which faces the wearer when in use.

As used herein, the term “longitudinal axis” refers to the imaginary line running through the center of the diaper bisecting the back waist region, the core, and the front waist region.

As used herein, “wearer” or “user” refers to the individual that will or is wearing the disposable absorbent garment. Exemplary embodiments of wearers are babies and toddlers, minors and adults. “Care taker” refers to an individual that puts on and/or removes the garment from the wearer. The care taker may also be a wearer.

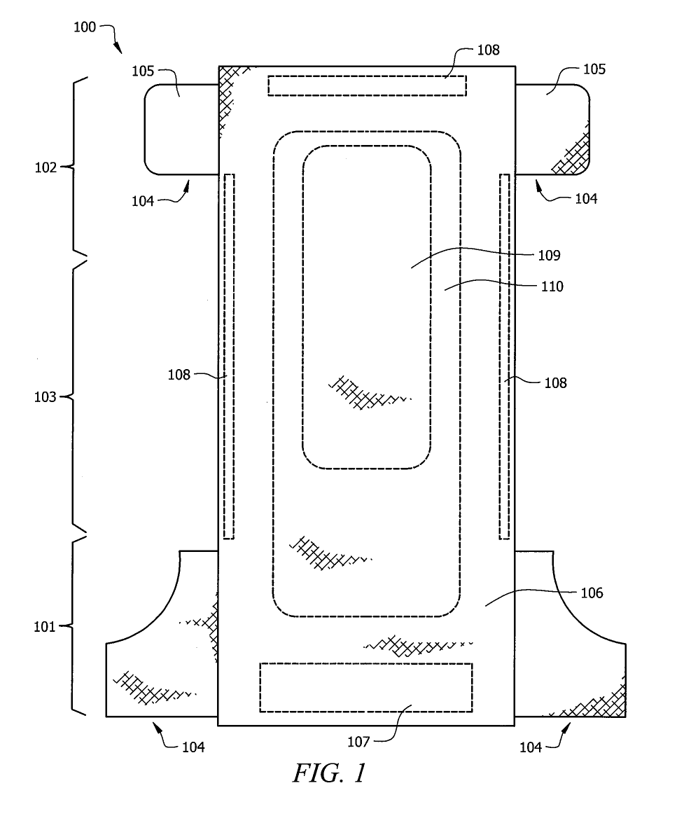

FIG. 1 illustrates a disposable absorbent garment 100 in accordance with an embodiment of the present application. The disposable absorbent garment 100 is of a type that can be placed against or in proximity to the body of a wearer so as to absorb and contain various bodily exudates. The embodiment of FIG. 1 is introduced to illustrate some basic features of a disposable diaper, most of which are also applicable to other disposable absorbent garments contemplated by the present application. Garment 100 is shown in the unfastened configuration. Disposable garment 100 includes three main regions aligned along an imaginary longitudinal axis. These regions include a first waist region 101, typically located at the front of the user when the garment is worn, a back waist region 102, and a crotch region 103. The shape of garment 100 may vary, and includes a generally rectangular shape, a T style shape, an I style shape, an hourglass shape, a combination of these shapes, or the like.

Disposable garment 100, at the front 101 and back 102 waist regions may include ear regions or protrusions 104 extending generally perpendicular to the longitudinal axis at waist regions 101 and 102. In this way, the front 101 and back 102 waist regions may be brought together and attached at the ear region to form holes to accommodate the wearer's legs. The shapes and configurations of ear portions 104 may be formed in any manner to accomplish the particular purposes of disposable garment 100. For example, ear portions 104 may be configured to be permanently or refastenably attached. Additionally, ear portions 104 may be known as side panels.

Disposable garment 100 also comprises fastener means 105 to attach the front 101 and back 102 waist portions of the garment together, to form an opening for the leg of the wearer. These fasteners 105 may be integrated onto the backsheet or topsheet 106, or may be attached to the topsheet or backsheet (not shown) as additional tabs. Examples of fasteners 105 may include hook and loop fasteners, buttons, pins, snaps, adhesive tape fasteners, cohesives, mushroom and loop fasteners, or the like.

More specifically, loop type fasteners typically comprise a fabric or material having a base or backing structure and a plurality of loop members extending upwardly from at least one surface of the backing structure. The loop material can be formed of any suitable material, such as acrylic, nylon or polyester, and can be formed by methods such as warp knitting, stitch bonding or needle punching. Suitable loop materials are known in the art and are available, for example, from manufacturers such as 3M, St. Paul Minn. (designated as a KN series loop). The loop material can be an integral portion of the outer surface or inner surface of the side panel rather than a separated material or structure.

Hook type fasteners typically comprise a fabric or material having a base or backing structure and a plurality of hook members extending upwardly from at least one surface of the backing structure. In contrast to the loop type fasteners which desirably comprise a flexible fabric, the hook material advantageously comprises a resilient material to minimize unintentional disengagement of the fastener components as a result of the hook material becoming deformed and catching on clothing or other items. The term “resilient” as used herein refers to an interlocking material having a predetermined shape and the property of the interlocking material to resume the predetermined shape after being engaged and disengaged from a mating, complementary interlocking material. Suitable hook material can be molded or extruded of nylon, polypropylene or another suitable material. Suitable single-sided hook materials for fasteners 105 are known in the art and are available, for example, from manufacturers such as 3M, St. Paul Minn. (designated as a CPL series loop).

Fasteners 105 may be located at the front 101 or back 102 waist region of garment 100, or at both. For example, in the representative embodiment of FIG. 1, the fasteners are shown on the back waist region 102 and are configured to engage with ear portions 104 located on the front waist region 101 of disposable garment 100. In other embodiments, a hook(s) may be disposed on the back waist region 102 with a corresponding loop(s) on front waist region 101.

Additionally, absorbent garment 100 may comprise elastic portions to help conform the absorbent diaper to the wearer's body. For example, generally some part of the waist region comprises elastic material 107 to fit the garment to the wearer's waist. Also, both sides of the crotch region 103 may comprise elastic material 108 to fit the garment to the wearer's legs, and to provide sealing means to keep any extra liquid or exude inside of the diaper. Suitable material for use as elastic members are well known to those skilled in the art. An embodiment of elastic material construction is to attach sheets or strands of ribbons of polymeric, elastomeric material and adhere them to a sheet in the extended configuration. These elastic member may include polyurethane, synthetic rubber, natural rubber, or any combination thereof. Exemplary embodiments of elastic members that may be used with the present invention are given in U.S. Pat. Nos. 4,892,536 issued Jan. 9, 1990 to DesMarais et al., 5,540,796 issued Jul. 30, 1996 to Fries, 4,895,568 issued Jan. 23, 1990 to Enloe, and 5,196,000 issued Mar. 23, 1993 to Clear et al.

The diaper may additionally include cuffs or flaps that run along the longitudinal axis close to the leg edge of the diaper. These cuffs provide extra sealing to keep excess fluids and material inside of the diaper. The cuffs may have additional elastic, e.g., elastic 108, in them to better fit the wearer, and may also comprise absorbent material to prevent leakage. Exemplary embodiments of cuffs are given in U.S. Pat. Nos. 4,704,116 issued Nov. 3, 1987 to Enloe, 5,582,606 issued Dec. 10, 1996 to Bruemmer et al., 4,795,454 issued Jan. 3, 1989 to Dragoo, and 5,085,654 issued Feb. 4, 1992 to Buell.

Absorbent garment 100 also includes acquisition distribution layer (ADL) 109. ADL 109 is generally a nonwoven sublayer placed between topsheet 106 and absorbent core 110. ADL 109 functions to distribute liquid over a larger surface area in order to increase saturation capacity of garment 100. Absorbent core 110 in accordance with embodiments of the present application comprises unbleached pulp along with other absorbing materials. The constituent parts of garment 100, and specifically absorbent core 110, are described in detail below.

The various components of the diaper, e.g., topsheet 106, ADL 109, absorbent core 110, elastic portions 107, 108, and ear portions 104, etc., are integrally assembled together using various types of suitable attachment means that are well known in the art. These attachments means include the non-limiting examples of adhesives, sonic boding, thermal boding, or any combination thereof.

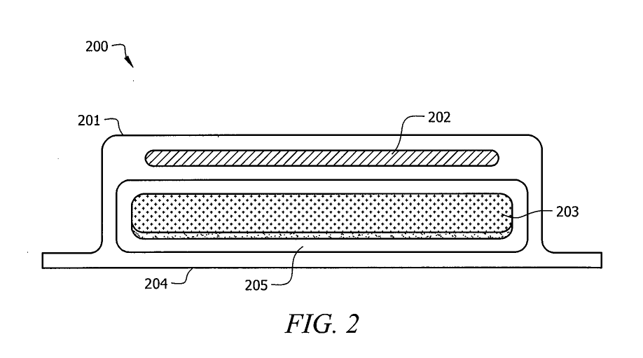

FIG. 2 illustrates a cross-section of one embodiment of an absorbent diaper 200 taken through the crotch region. This cross-section comprises from the top of the drawing to the bottom, a topsheet 201, an ADL 202, an absorbent core 203, and the backsheet 204. Additionally, as depicted in FIG. 2, diaper 200 may also include a tissue or nonwoven cover 205 above and/or below absorbent core 203. Nonwoven cover 205 provides containment of the core materials preventing migration of fines and particulates during manufacturing and use and acts as a barrier preventing direct contact of the core and the wearer's skin. Cover 205 may also provide additional structural stability to the core.

The backsheet 204 is generally that portion of the diaper 200 positioned adjacent the garment facing surface of the absorbent core 203 which prevents the exudates absorbed and contained therein from soiling articles which may contact the diaper 200, such as bedsheets, clothes and undergarments. In preferred embodiments, the backsheet 204 is impervious to liquids (e.g., urine) and comprises a thin plastic film such as a thermoplastic film having a thickness of about 0.012 mm (0.5 mils) to about 0.051 mm (2.0 mils). Suitable backsheet films include those manufactured by Tredegar Industries Inc. of Terre Haute, Ind. and sold under the trade name X15306, X10962 and X10964. Other suitable backsheet materials may include breathable materials which permit vapors to escape from the diaper 200 while still preventing exudates from passing through the backsheet 204. Exemplary breathable materials may include materials such as woven webs, nonwoven webs, composite materials such as film-coated nonwoven webs, and macroporous films such as manufactured by Mitsui Toatsu Co. of Japan under the designation ESPOIR NO and by Tredegar Corp. of Richmond, Va. under the designation EXAIRE. Suitable breathable composite materials comprising polymer blends are available from Clopay Corporation, Cincinnati, Ohio under the Name HYTREL blend P18-3097.

Backsheet 204 may be joined to topsheet 201, absorbent core 203 or any other element of the diaper 200 by any attachment means known in the art. For example, the attachment means may include a uniform continuous layer of adhesive, a patterned layer of adhesive, or an array of separate lines, spirals, or spots of adhesive. One preferred attachment means comprises an open pattern network of filaments of adhesive as disclosed in U.S. Pat. No. 4,573,986 issued Mar. 4, 1986 to Minetola et al. Other suitable attachment means include several lines of adhesive filaments which are swirled into a spiral pattern, as is illustrated by the apparatus and methods shown in U.S. Pat. Nos. 3,911,173 issued Oct. 7, 1975 to Sprague, Jr., 4,785,996 issued Nov. 22, 1978 to Ziecker, et al., and 4,842,666 issued Jun. 27, 1989 to Werenicz. Each of these patents are incorporated herein by reference. Adhesives which have been found to be satisfactory are manufactured by H. B. Fuller Company of St. Paul, Minn. and marketed as HL-1620 and HL-1358-XZP. Alternatively, the attachment means may comprise heat bonds, pressure bonds, ultrasonic bonds, dynamic mechanical bonds, or any other suitable attachment means or combinations of these attachment means as are known in the art.

Topsheet 201 is preferably positioned adjacent to the body of a user and between the user and absorbent core 203. Topsheet 201 may be joined to absorbent core 203 and/or to backsheet 204 by any attachment means suitable for such joining, such as discussed above. In some embodiments, topsheet 201 and backsheet 204 are joined directly in some locations and indirectly joined in other locations by directly joining topsheet 201 to other elements of diaper 200.

The topsheet 201 is preferably compliant, soft feeling, and non-irritating to the wearer's skin. Further, at least a portion of the topsheet 201 is liquid pervious, permitting liquids to readily penetrate through its thickness. A suitable topsheet 201 may be manufactured from a wide range of materials, such as porous foams, reticulated foams, apertured plastic films, or woven or nonwoven webs of natural fibers (e.g., wood or cotton fibers), synthetic fibers (e.g., polyester or polypropylene fibers), or a combination of natural and synthetic fibers. If the absorbent assemblies include fibers, the fibers may be spunbond, carded, wet-laid, meltblown, hydroentangled, or otherwise processed as is known in the art. For example, one suitable topsheet 201 comprising a web of staple length polypropylene fibers is manufactured by Veratec, Inc., a Division of International Paper Company, of Walpole, Mass. under the designation P-8.

Preferably, topsheet 201 is made of a hydrophobic material or is treated to be hydrophobic in order to isolate the wearer's skin from liquids contained in absorbent core 203. If topsheet 201 is made of a hydrophobic material, preferably at least the upper surface of topsheet 201 is treated to be hydrophilic so that liquids will transfer through the topsheet more rapidly. This diminishes the likelihood that body exudates will flow off of topsheet 201 rather than being drawn through the topsheet 201 and being absorbed by the absorbent core 203. Topsheet 201 can be rendered hydrophilic by treating it with a surfactant or by incorporating a surfactant into the topsheet. Suitable methods for treating topsheet 201 with a surfactant include spraying the topsheet 201 material with the surfactant and immersing the material into the surfactant. A more detailed discussion of such a treatment and hydrophilicity is contained in U.S. Pat. No. 4,988,344 issued Jan. 29, 1991 to Reising, et al. and U.S. Pat. No. 4,988,345 issued Jan. 29, 1991 to Reising. A more detailed discussion of some suitable methods for incorporating surfactant in the topsheet can be found in U.S. Statutory Invention Registration No. H1670, published on Jul. 1, 1997 in the names of Aziz et al. Each of these references is hereby incorporated by reference herein. Alternatively, the topsheet 201 may include an apertured web or film which is hydrophobic. This may be accomplished eliminating the hydrophilizing treatment step from the production process and/or applying a hydrophobic treatment to topsheet 201, such as a polytetraflouroethylene compound like SCOTCHGUARD or a hydrophobic lotion composition, as described below. In such embodiments, it is preferred that the apertures be large enough to allow the penetration of aqueous fluids like urine without significant resistance.

Any portion of the topsheet 201 may be coated with a lotion as is known in the art. The lotion may function alone or in combination with another agent as the hydrophobizing treatment described above. Topsheet 201 may also include or be treated with antibacterial agents. Further, the topsheet 201, the backsheet 204 or any portion of the topsheet 201 or backsheet 204 may be embossed and/or matte finished to provide a more cloth like appearance.

The illustrated embodiment of FIG. 2 includes ADL 202. ADL 202 may also be known as the surge management layer. ADL 202 incorporates materials that are designed primarily to receive, temporarily store, and/or distribute liquid along the mutually facing surface with absorbent core 203, thereby maximizing the absorbent capacity of absorbent core 203. One suitable material for ADL 202 comprises a material having a basis weight of about 50 grams per square meter, and comprising a through-air-bonded-carded web of a homogeneous blend of 60% 3 denier bicomponent fiber comprising a polyester core/polyethylene sheath, commercially available from BASF Corporation, and 40% 6 denier polyester fiber, commercially available from Hoechst Celanese Corporation. Additionally, ADL 202 may comprise any of or combination of, and not limited to, spun, carded, meltblown, wetlaid, hydroentangled fibers or apertured film.

Absorbent core 203 may be comprised of a variety of different materials. It may be constructed from materials such as hydrophilic fibers, cellulosic fluff such as wood pulp, superabsorbent hydrogel forming particles, synthetic polymeric, meltblown fibers, natural fibers, or combinations thereof. Example embodiments utilize unbleached pulp in absorbent core 203 and/or a combination of bleached and unbleached pulp. Further, absorbent core 203 may be made from a uniform mixture of materials, or may be non-uniformly mixed.

Example superabsorbent materials can be selected from natural, synthetic, and modified natural polymers and materials. The superabsorbent materials can be inorganic materials, such as silica gels, or organic compounds, such as crosslinked polymers. Suitable superabsorbent materials are available from various commercial vendors, such as BASF located in Charlotte, N.C., and Stockhausen GmbH & Co. KG, D-47805 Krefeld, Federal Republic of Germany. Typically, a superabsorbent material is capable of absorbing at least about 15 times its weight in water, and desirably is capable of absorbing more than about 25 times its weight in water. In some embodiments, it may be preferred to utilize a superabsorbent material with a high pressure absorbency index (PAI) such as described in U.S. Pat. No. 5,601,542 Feb. 11, 1997 to Melius et. al. Current implementations utilize materials that have an approximate 120 PAI rating. However, other embodiments may utilize a material having 10 PAI or higher.

In accordance with embodiments of the present application, absorbent core 203 comprises of about 40% to 80% superabsorbent polymer and anywhere from 60% to 20% of the unbleached pulp. Example embodiments utilize UB Kraft Pulp from International Paper Corporation to implement the unbleached pulp. It is noted that such a material is not normally utilized in the absorbent garment industry due to properties which are perceived as not conducive to effective absorbent garment manufacturing. Some of these properties are discussed below.

It is noted that embodiments may utilize varying percentages and types of pulp products, superabsorbent products for absorbent core 203. For example, some embodiments may utilize only unbleached pulp, while others may utilize unbleached pulp as a percentage of the fiber content. Specifically, in one embodiment, the unbleached pulp content of absorbent core 203 may be as low as 25% of the fiber content and then the remaining 75% of the fiber content would be either a bleached, totally chlorine-free (TCF) or an elemental chlorine-free (ECF) pulp. Further embodiments may vary as to the proportions of pulp and superabsorbent materials. Specifically, one embodiment utilizes 55% superabsorbent materials and 45% pulp content. It is noted that this amount of superabsorbent material is a much higher percentage than has typically been used in this type of a product in the past (with previous products generally staying below 45% superabsorbent materials in an absorbent core). This amount of super absorbent material is able to be utilized due to the distributive qualities of the pulp of various embodiments.

Unbleached pulp, in accordance with some embodiments, may include a mixture of softwood and hardwood fiber content. Hardwood fibers generally provide much shorter and less coarse fiber with respect to softwood fibers. Such fibers are generally not favored with hygiene pulp manufacturers, and in fact, these fibers are often referred to as “trash” because of their appearance and performance. However, it has been discovered that these shorter fibers may assist in providing a degree of polymer separation and fiber separation and also open up capillary spacing in absorbent core 203. Whereas in the past, shorter fibers were perceived as being an undesirable in the product because did not have the desired capillary properties. In any event, the inventors have discovered that in present unbleached pulp embodiments utilizing predominantly softwood fibers, some amount of hardwood fibers does not meaningfully negatively affect the performance of absorbent core 203. For example, in some embodiments the unbleached pulp may have as much as 60% hardwood fibers. Because of this, processing steps in making the unbleached pulp are reduced as manufacturers are not forced to utilize processing steps which segregate softwood from hardwood, thereby reducing manufacturing costs.

In some embodiments, unbleached pulp utilized in absorbent core 203 may also contain various constituents as a result of forgoing the normal bleaching processes. Such constituents have previously been considered harmful to the performance of an absorbent garment. For example, unbleached pulp will generally have a much higher content of lignin (e.g. greater than 10 times the amount), hemicelluloses, and other organics than would be present in respective bleached materials. By having some of these other constituents present, the contact angle changes and the surface texture of the fiber changes which is another reason why the absorbent garment industry has seen these constituents as harmful to the performance of the garment. However, when utilizing unbleached pulp materials having these constituents, along with a high concentration of superabsorbent polymers, present embodiments yield products which have exceeded the performance of counterparts that utilize a totally chlorine-free bleached pulp in an absorbent core. As such, embodiments may also reduce the amount of processing steps and chemicals necessary for manufacturing pulp because additional steps to remove or reduce these constituents may be minimized, while maintaining acceptable performance properties for an absorbent garment.

As can be seen, utilizing unbleached pulp having the above-noted additional constituents and differences in fiber length changes the surface properties of the pulp itself. For example, unbleached pulp tends to have different texture and contact angle properties. The differing texture and contact angle tends to change the distribution properties of fluids and to also yield a slightly rougher surface. Additionally, with the presence organics, the pulp tends to stick together better in order to provide improved structural integrity.

As discussed above, manufacturing unbleached pulp yields a significantly simpler process as opposed to pulp which undergoes a bleaching process. Additionally, the utilization of unbleached pulp in accordance with embodiments of the present application may also assist in simplifying the absorbent garment manufacturing process. For example, as will be discussed below, the usage of unbleached pulp has yielded improved fiberisation and formation of the absorbent core, requires less energy to process and reduces static charge generation.

In a typical core formation process, a pulp sheet will be processed through a hammer mill. This mill impacts the surface along the edge of the pulp sheet that is being processed and the impact separates the fibers out into individual fibers. When processing a pulp sheet, there are a certain percentage of “knots” and a certain percentage of “fines” that are present. Knots are undesirable because they represent an unfiberized portion of a pulp sheet. Fines are generally perceived as dust, rather than actual fibers, which are also not desirable. It is desirable to have a majority of the pulp sheet classified as “accepts,” which are full length fibers that have not been broken apart. It has been found that when utilizing unbleached pulp, the number of knots is very minimal. Because this material fiberizes in such a manner, it is possible to decrease the amount of energy consumption during the fiberisation phase by reducing the energy required by the hammer mill.

The actual formation of the core is also improved when utilizing unbleached pulp materials. For example, once the fiberized material leaves the hammer mill, it goes into a vacuum forming process where fibers are drawn into a large chamber and down onto a screen. Because the previous process has improved fiberisation, the fibers tend to stay entrained in the air flow of this process, which tends to yield more uniform formed pad.

Another improvement observed which results from an increase in sugar and organic content within the unbleached pulp material is that less static electricity is generated during the transportation phase from the hammer mill to the forming section. Static buildup is caused as a result of materials colliding and brushing against, each other and the surfaces confining and facilitating the forming process. Static charges are induced by the superabsorbent materials and by fibers inside the forming section where the absorbent materials can accumulate into a mass, adhering to surfaces inside the forming area. The materials generally build to a point where the static charge can no longer support adhesion to the surface and the mass sloughs off and drops into one of the forming pockets. The irregularity of buildup and slough off may cause irregularity in the pad. However, the sugars and organics from the unbleached pulp material may be utilized to dissipate some of that static charge which may also lead to the improved formation.

As discussed throughout the present application, the performance of the end product of these processes, the disposable absorbent garment, is improved by the use of unbleached pulp materials in the absorbent core. For example, the improved contact angle, the sugars, organics and hardwood content help in the distribution of liquids and functions to create separation between the superabsorbent particles. Specifically, the separation between the superabsorbent particles and separation between the fibers help to maintain capillary spacing that will then promote the distribution of the fluid. It is further noted that better distribution will also reduce the incidence of leakage whereas a larger volume of the absorbent core will be utilized during use.

Other properties of the end product may also be useful. For example, because there is a brown fiber content in the absorbent core, the end product takes on a light tan color. As noted above, such a tan color in most applications or products is seen as unmarketable for appearance sake. However, because of the unbleached materials, when the product is in use and it is wetted, the fibers appear much darker. Once the fibers are wetted, the surface color at the backsheet changes as the fibers come in contact with one another. As a result, the appearance of the core through the backsheet becomes much darker at the point of insult or where the article is wet. As a result, the diaper can actually give an initial indication that that diaper has been wetted. Over a period of time and as the fluid from the wetting dissipates and gets absorbed by the superabsorbent material, it will revert to a light tan color until the pulp, finally, through multiple wettings, reaches a saturation point and the superabsorbent reaches a saturation point and the fibers stay wet.

Additionally, as discussed above, as a result of the fibers having a softwood content and because there are sugars and organics present (e.g., lignin), the surface of the fibers have increased tack or stickiness. As such, when the fibers are laid down in the manufacturing process, these properties increase the coefficient of friction and improve properties achieved through fiber entanglement. This creates a core that is less susceptible to separating and therefore improves the structural integrity of the absorbent garment.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A disposable absorbent article having a front waist region, a back waist region, and a crotch region located between the front and back waist regions, the disposable article comprising:
a liquid pervious topsheet;
a liquid impervious backsheet; and
an absorbent core disposed between at least a portion of the topsheet and bottom sheet, wherein the absorbent core comprises a superabsorbent material and pulp material, wherein at least a portion of the pulp material includes unbleached fibers.

2. The disposable absorbent article of claim 1 wherein the unbleached fiber comprises one or more constituents which alter the contact angle properties of the absorbent core.

3. The disposable absorbent article of claim 1 wherein the unbleached fiber comprises one or more constituents which alter the fiber texture of the absorbent core.

4. The disposable absorbent article of claim 1 wherein the unbleached fiber comprises one or more constituents which increase the structural integrity of the absorbent core.

5. The disposable absorbent article of claim 1 wherein the unbleached fiber comprises one or more constituents which increase capillary forces within the absorbent core.

6. The disposable absorbent article of claim 1 wherein the unbleached fiber comprises one or more constituents selected from a list comprising lignin, hemicelluloses, sugars and hardwood fibers.

7. The disposable absorbent article of claim 1 wherein a change in color of the absorbent core provides temporary visual indication of wetness.

8. The disposable absorbent article of claim 1 wherein the unbleached fibers comprise softwood and hardwood fibers.

9. The disposable absorbent article of claim 8 wherein the softwood fibers are less coarse than the hardwood fibers.

10. The disposable absorbent article of claim 8 wherein the hardwood fibers comprise up to 60% of the pulp material.

11. The disposable absorbent article of claim 1 wherein at least a portion of the pulp material comprises bleached fibers.

12. The disposable absorbent article of claim 1 wherein the superabsorbent material comprises greater than 40% of the absorbent core materials.

13. A disposable absorbent article having a front waist region, a back waist region, and a crotch region located between the front and back waist regions, the disposable article comprising:
a topsheet;
a backsheet; and
an absorbent core disposed between at least a portion of the topsheet and bottom sheet, wherein the absorbent core comprises a superabsorbent material and pulp material, wherein at least a portion of the pulp material includes unbleached fibers and at least one of bleached or semi-bleached fibers.

14. The disposable absorbent article of claim 13 wherein the pulp material comprises at least 10% unbleached fibers.

15. The disposable absorbent article of claim 13 wherein the pulp material comprises at least 80% unbleached fibers.

16. The disposable absorbent article of claim 13 wherein unbleached fibers comprise increased lignin content, sugar content and organic content with respect to that of the bleached fibers.

17. A method of making a disposable absorbent article having a front waist region, a back waist region, and a crotch region located between the front and back waist regions, the method comprising:
forming an absorbent core, the absorbent core comprising a majority part superabsorbent material and a minority part pulp material wherein the pulp material includes unbleached fibers;
placing the absorbent core between a liquid pervious topsheet and a liquid impervious backsheet; and
coupling the topsheet and backsheet along the front waist region, back waist region and crotch region.

18. The method of claim 17 wherein the pulp material comprises only unbleached fibers.

19. The method of claim 17 wherein the pulp material comprises bleached and unbleached fibers.

20. The method of claim 17 wherein the pulp material comprises semi-bleached and unbleached fibers.
